(19) United States
(12) Patent Application Publication
(10) Pub. No.: US 2008/0071223 A1
(43) Pub. Date: Mar. 20, 2008

(54) STEERABLE SURGICAL GUIDE WIRE INTRODUCER

(75) Inventor: Marshall Ephraim Stauber, Hollywood, FL (US)

Correspondence Address: INVENTIT, LLC, 3702 WASHINGTON STREET Suite 101, HOLLYWOOD, FL, 33021, US

(73) Assignee: INVENTIT, LLC, Hollywood, FL (US)

(21) Appl. No.: 11/309,701

(22) Filed: Sep. 15, 2006

(51) Int. Cl. A61M 5/178

(52) U.S. Cl. 604/164.13

(57) ABSTRACT

A Guide Wire Introducer with integrated wire steering function for treating bone. The guide wire employed with the introducer consists of a core material that is a psuedoelastic metal alloy, such as Nitinol. This introducer has a chamfered tip with the complement of the outer construction consisting of a uniform narrow outer diameter enabling insertion into soft tissue and bone with a guide wire within. The chamfered tip of the introducer incorporates an integrated slit that enables the steering function of the introducer. Additionally, the outer surface incorporates a radiolucent notch for radial position reference.

### FIELD OF INVENTION

This invention relates to the field of medical devices that are used for enabling percutaneous minimally invasive access for the treatment of cancellous bone using guide wire directed devices.

### BACKGROUND OF THE INVENTION

Guide wires can be used in a range of surgical procedures to reduce the invasiveness of the respective procedure. For example, there are surgical procedures to correct injuries and maladies of the bone through the introduction of a flexible to semi-rigid/rigid catheter or tube that can be guided by a surgical guide wire. In a specific example, this invention can be employed in the surgical correction of a vertebral compression fracture injury that can be treated with bone cement. In this specific example, a semi-rigid tube or catheter is introduced into the volume of the compression fracture of the vertebral body and a balloon or bladder is introduced through a catheter or tube into the cancellous bone and inflated to correct the kyphosis induced by the compression fracture of a vertebral body. Afterwards the bladder or balloon is removed, and then bone cement is introduced using a catheter or tube. The guidance of catheter(s) or tube(s) in the example above is critical, and can be obtained by employing the use of a guide wire that has been introduced and steered to the optimal location by the provided invention.

It is the object of this invention to enable or enhance procedures that would benefit from the use of a steerable guide wire with emphasis on procedures involving the guidance of a catheter or tube into cancellous bone. Further objects and advantages of the provided invention will become apparent from a consideration of the drawings and ensuing description.

### SUMMARY OF THE INVENTION

The provided invention consists of a Guide Wire Introducer with integrated wire steering function for treating bone. The introducer is designed to employ a guide wire with a core material that behaves like a psuedoelastic metal alloy, such as Nitinol. This introducer is tubular and rigid with a chamfered tip at its distal end that enables introduction into soft tissue, and limited introduction into bone. The majority of the tubular introducer has a maximum outer diameter that is approximately three millimeters in diameter, for the typical vertebral body application. The exact maximum outer diameter for the introducer varies minutely according to specific application. Most of the outer surfaces are the complement of the outer construction other than the chamfered tip at the distal end, and consist of the uniform narrow outer diameter indicated above, a characteristic that enables insertion into soft tissue and bone with a guide wire within the introducers' inner tubular portion. Within the chamfered tip of the introducer and incorporated into the body of the introducer is an integrated slit that enables the steering function of the introducer by entrapping the guide wire. Within four to eight millimeters of the slit is a radiolucent notch incorporated within the outer surface, for radial position referencing that assists the surgeon in determining the radial position of the slit.

### BRIEF DESCRIPTION OF DRAWINGS

Figures

FIG. 1 shows the entire surgical guide wire introducer with the integrated slit and radiolucent notch exposed.

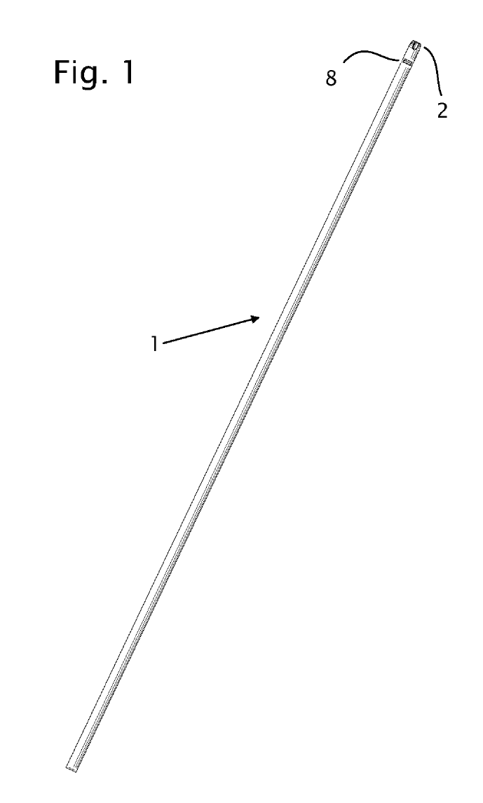

FIG. 2 shows the extent of the enlarged partial view seen in FIG. 3.

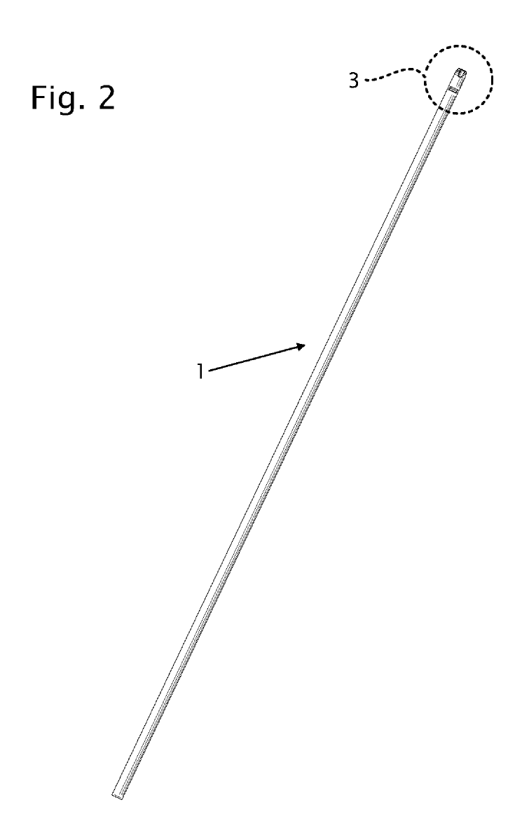

FIG. 3 is an enlarged view of the radiolucent referencing notch and the introducer integrated slit.

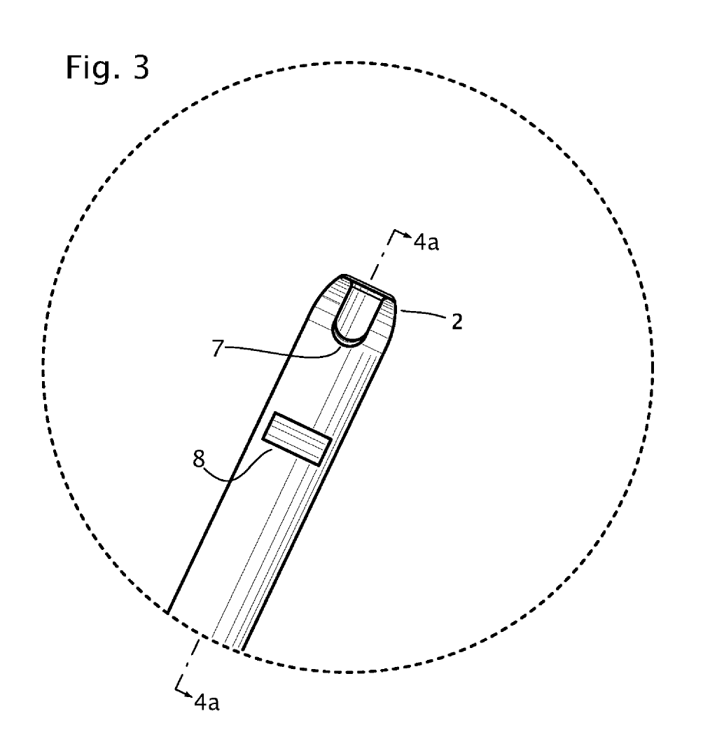

FIG. 4a is a sectional view of FIG. 3 showing a profile view of the radiolucent notch. In addition, the bore of the instrument and the integrated slit are also shown.

FIG. 4b is a sectional view of FIG. 3 showing a profile view of the radiolucent notch with a surgical guide wire shown in the bore of the instrument. This illustration shows a view of a surgical guide wire before it is advanced to the extent were its strategic bend is within range of the integrated slit.

FIG. 4c is a sectional view of FIG. 3 showing a profile view of the radiolucent notch with a surgical guide wire shown in the bore of the instrument. This illustration shows a view of a surgical guide wire as it is advanced to the extent were its strategic bend slides into the integrated slit.

FIG. 5 is a simplified kyphoplasti example illustrating the application of the guide wire introducer in a vertebral application.

FIG. 6 is a simplified kyphoplasti example illustrating the application of the guide wire introducer in a vertebral application with a surgical guide wire being introduced through its distal tip.

### DRAWINGS—REFERENCE NUMERALS

Listing 1, 2, 7, 8, 9, 10, 11

| | | | |
|---|---|---|---|
| 1 | Steerable Surgical Guide Wire Introducer. | 2 | Chamfered tip. |
| 7 | Integrated slit. | 8 | Radial |
| | | | referencing notch. |
| 9 | Guide Wire inserted into introducer. | 10 | Guide Wire bend |
| | | | entrapment within |
| | | | slit |
| 11 | Simplified representation of a vertebra with | | |
| | Steerable Surgical Guide Wire Introducer. | | |

### DETAILED DESCRIPTION

The embodiments of the provided invention are employable in the applications as indicated above in addition to all those that can be commonly inferred from those indicated above. It is the object of this invention to enable or enhance procedures that would benefit from the use of a steerable guide wire 9 with emphasis on procedures involving the guidance of a surgical guide wire that has been introduced and advanced into cancellous bone.

In the provided invention, the chamfered tip of the introducer incorporates an integrated slit 7 that enables the steering function of the introducer. This introducer 1 has a chamfered tip 2 with the complement of the outer construction consisting of a uniform narrow outer diameter of approximately 3 mm for the typical application, enabling insertion into soft tissue and to a limited extent in bone, with a guide wire within its inner tubular cavity. Additionally, the outer surface incorporates a radiolucent notch 8 for radial position reference.

Suitable material such as surgical stainless steel or similar material is desired in the production of the preferred embodiment of the introducer to provide for the desired strength, flexibility, and fatigue life.

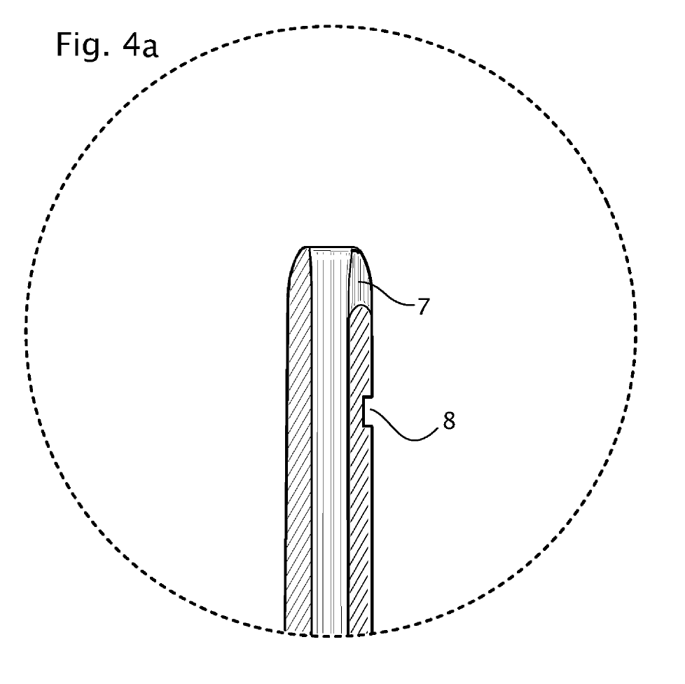

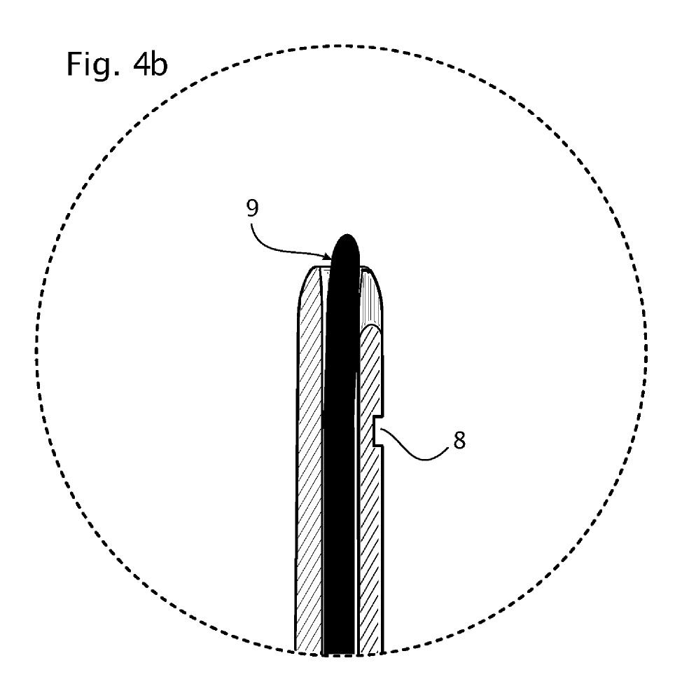

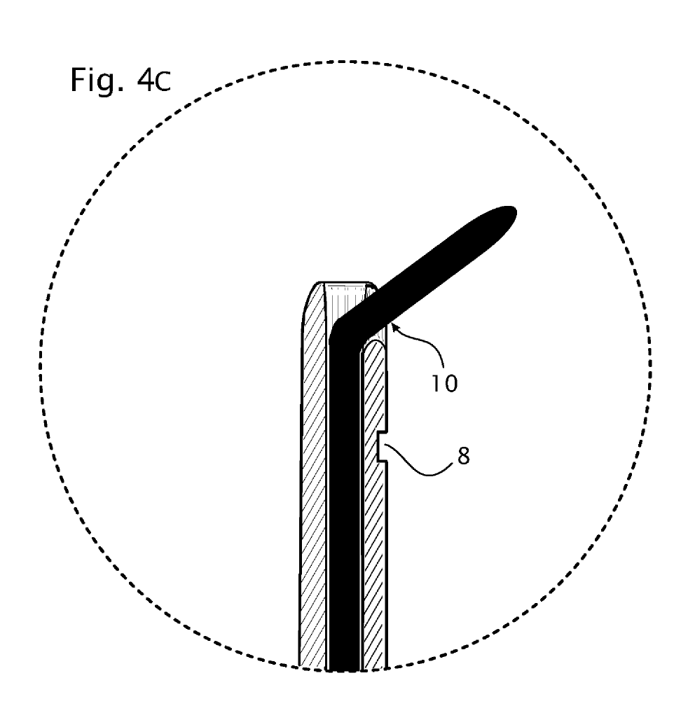

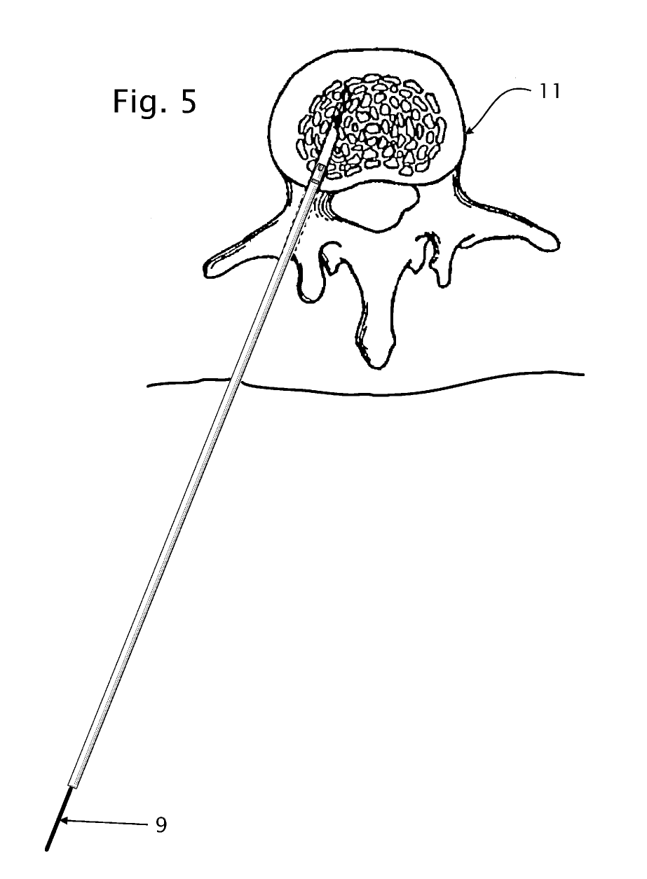

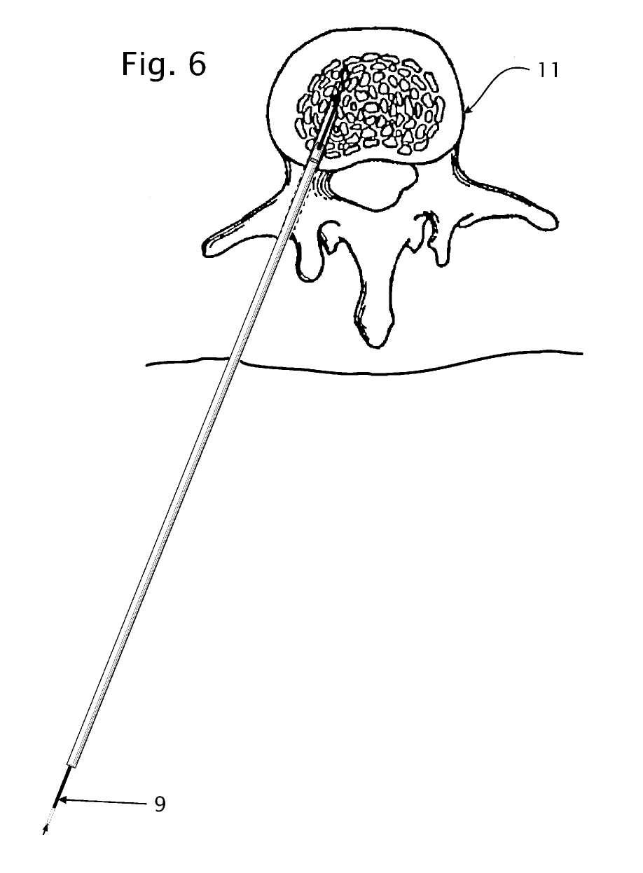

The provided Guide Wire Introducer with integrated wire steering function for treating bone, is shaped as a long thin tubular structure 1 with a psuedoelastic surgical guidewire 9 carried within. The guide wire 9 employed with the introducer consists of a core material that is a psuedoelastic metal alloy, such as Nitinol or similar material. When employed in the treatment of the vertebral body for example, a pilot hole may or may not be drilled depending on the approach to the vertebral body and condition of the vertebral body during the procedure when the provided invention is employed. Within a preferred application such as the correction of compression fracture induced kyphosis, the introducer 1 is placed {after formation of a pilot hole if necessary} with the guide wire 9 within and not protruding markedly beyond the chamfered tip 2. The guide wire placed within the introducer will have preformed bends in strategic directions and length intervals 10 to allow the guide wire to slip into the chamfered tip integrated slit 7 as these strategic intervals of the guide wire are advanced within range of the integrated slit 7, thus allowing radial steering function when the guide wire slips within the slit after a strategic interval of axial advancement, as indicated in FIGS. 4b and 4c. During such strategic intervals of axial advancement of the guide wire, as a strategically placed preformed bend in the guide wire advances 0.5 to 1 mm proximal to the integrated slit 7, the guide wire then becomes entrapped within the slit of the chamfered tip. This occurrence of guide wire entrapment enables radial manipulation by the introducer in a radial clockwise or counter clockwise fashion—via rotation of the introducer about its long axis. At this point of guide wire advancement, the introducer tip integrated slit 7 will continue to entrap the guide wire to enable radial manipulation until the wire is either advanced or retracted from its current position within the introducer or forced out of the tip slit 7. After subsequent axial advancement or retraction of the guide wire within the introducer, the radial steering function of the introducer tip is then reduced or disabled as the guide wire bend moves beyond the position of the integrated slit 7. A illustration of the introducer implementation can be seen in the simplified kyphoplasti example shown in FIGS. 5 and 6.

In addition to the specific function/uses indicated above, the device described herein would also be well suited for use in treating and/or reinforcing weakened, diseased and/or fractured bones in various locations throughout the body. For example, the disclosed device could be used to facilitate the delivery of reinforcing materials and/or medications, such as cancer drugs, replacement bone cells, collagen, bone matrix, demineralized calcium, and other materials/medications, directly into a fractured, weakened and/or diseased bone, thereby increasing the efficacy of the materials, reinforcing the weakened bone and/or speed healing. Moreover, using the steerable guide wire introducer to facilitate targeted injection of such materials into one bone within a body could permit the medication/material to migrate and/or be transported to other bones and/or organs in the body through strategically locating guide wire apparatus, thereby improving the quality of bones and/or other organs not directly injected with the materials and/or medications.

Other embodiments and uses of the invention will be apparent to those skilled in the art from consideration of the specification and practice of the invention disclosed herein. All documents referenced herein are specifically and entirely incorporated by reference. The specification and example should be considered exemplary only, with the true scope and spirit of the invention indicated by the following claims. As will be easily understood by those of ordinary skill in the art, variations and modifications of each of the disclosed embodiments can be easily made within the scope of this invention as defined by the following claims.

What is claimed is:

1. A tubular device arranged to be used as a surgical guide wire introducer comprising:
a tubular structure having a chamfer on its distal end;
the chamfer of said tubular structure having a slit thereupon.

2. The tubular device of claim 1 wherein said slit provides an approximate interference fit within its gap for a given surgical guide wire used within the device, thereby providing a means for entrapping the given surgical guide wire used within the device when the surgical guide wire is bent to a radius that enables the given surgical guide wire within the device to slidably engage the gap within said slit when the given surgical guide wire has been advanced or retracted through the tubular device until the bent portion of the given surgical guide wire approximately intersects with said slit, whereby this engagement of the given surgical guide wire within said slit enables radial manipulation of the surgical guide wire used within the device.

3. The tubular instrument of claim 1 having a radiolucent alteration on its exterior surfaces thereof substantially similar to a notch, to provide a means for discerning the radial and axial orientation of the instrument.
